(19) United States
(12) Patent Application Publication
(10) Pub. No.: US 2005/0033324 A1
(43) Pub. Date: Feb. 10, 2005

(54) Surgical tissue guard

(75) Inventor: Hai N. Phan, Lake Forest, CA (US)

Correspondence Address: KNOBBE MARTENS OLSON & BEAR LLP, 2040 MAIN STREET, FOURTEENTH FLOOR, IRVINE, CA, 92614, US

(73) Assignee: Dexteus

(21) Appl. No.: 10/890,974

(22) Filed: Jul. 14, 2004

Related U.S. Application Data
(60) Provisional application No. 60/487,006, filed on Jul. 14, 2003.

(51) Int. Cl. A61B 17/04

(52) U.S. Cl. 606/148

(57) ABSTRACT

A guard attachable to a forceps is provided. In one embodiment, the guard is releasably securable to one arm of the forceps. In another embodiment, the guard is permanently secured to or integral with one arm of the forceps. In another embodiment, the guard is rotatable with respect to the forceps. A barrier portion of the guard extends between the forceps' arms, or along an outside of the forceps' arms. The barrier prevents tissue from getting between the forceps' arms. A physician can thus use the barrier to displace tissue from a surgical site. The barrier also provides protection to surrounding tissue, preventing suturing needles from pricking the tissue behind the barrier or pricking the physician. The guard may be slidable along at least a portion of the length of the forceps.

### RELATED APPLICATION

This application claims priority to provisional application Ser. No. 60/487,006 filed on Jul. 14, 2003, the entire contents of which are hereby expressly incorporated by reference.

### BACKGROUND OF THE INVENTION

1. Field of the Invention

The present invention relates to surgical instruments. More particularly, the present surgical tissue guard provides a barrier that enables a surgeon to displace tissue from a surgical site, and reduces the likelihood of tissue damage caused by accidental needle pricks during a suturing procedure.

2. Description of the Related Art

Surgeons typically use forceps during surgical procedures. A typical forceps comprises first and second grasping arms that are pivotably connected to one another at a first end. In a resting configuration the arms form a V. By squeezing the arms toward one another, the surgeon is able to grasp objects between the arms. When the surgeon removes the squeezing force, the arms return to their resting V configuration. Ends of the forceps may have sharp features that enhance the ability of the forceps to grasp tissue, which can sometimes be slippery.

Frequently, a physician will use forceps to reach into a surgical site and displace tissue in order to expose a particular area of tissue to be operated upon. Because of the configuration of the forceps, tissue often stubbornly intrudes into the area between the forceps' arms. This tissue obscures the tissue that the surgeon is trying to access. Sharp features on the forceps may damage this tissue. Therefore, a device that enables a surgeon to positively displace extraneous tissue while accessing other tissue would be of great benefit to surgeons.

Surgeons also frequently use forceps to grasp a suturing needle during a suturing procedure. A surgeon typically uses two forceps, pushing tissue out of the way with one forceps while grasping the needle with the other forceps and pushing the needle through the tissue being sutured. The sharp needle tip presents a significant danger to tissue surrounding the suture site, and to the surgeon's fingers. Therefore, a device that protects surrounding tissue and the surgeon's fingers during a suturing procedure would be of great benefit to both patients and surgeons.

U.S. Pat. No. 5,569,271 to Hoel discloses a surgical instrument for suturing and for manually manipulating suture needles. The device is formed of two elongate members hinged together. On one side of the hinge, the elongate members form a pair of jaws that are relatively moveable. Handles on the other end of the elongate members allow the user to manipulate the jaws about the hinge. An arcuate shield is integral with one of the jaws at its center. The shield extends laterally and arcuately from the jaw and covers the opposing jaw. The placement of the shield is such that a needle that is held between the jaws is protected from sticking anyone or anything. The jaw members may be made up of several units forming segmented jaws.

U.S. Pat. No. 4,776,791 to Hannula et al. discloses a shield for a pair of orthodontic pliers for use in removing dental brackets. The shield fits snugly onto one jaw of the pliers and provides side walls adjacent the jaws of the pliers. The jaws have a recess near their distal end such that the confronting surfaces of the jaw recesses and the confronting surfaces of the side walls provide an enclosure about a bracket while it is being removed. If the bracket fractures, the enclosure will contain any pieces that may break off the bracket.

### SUMMARY OF THE INVENTION

The preferred embodiments of the present surgical tissue guard have several features, no single one of which is solely responsible for their desirable attributes. Without limiting the scope of this surgical tissue guard as expressed by the claims that follow, its more prominent features will now be discussed briefly. After considering this discussion, and particularly after reading the section entitled “Detailed Description of the Preferred Embodiments,” one will understand how the features of the preferred embodiments provide advantages, which include retention of the full functionality of the forceps coupled with the capability to positively displace extraneous tissue while accessing other tissue, and protecting surrounding tissue and a surgeon's fingers during a suturing procedure.

A preferred embodiment of the present surgical tissue guard comprises a guard adapted to be attached to a forceps. The guard includes a barrier portion and at least one clip extending from the barrier portion. The clip includes first and second spaced side walls.

Another preferred embodiment of the present surgical tissue guard comprises a method of protecting tissue during a surgical or suturing procedure. The method comprises the steps of attaching to a forceps a tissue guard, and performing a surgical or suturing procedure. The guard includes a barrier portion and at least one clip extending from the barrier portion.

### BRIEF DESCRIPTION OF THE DRAWINGS

The preferred embodiments of the present surgical tissue guard, illustrating its features, will now be discussed in detail. These embodiments depict the novel and non-obvious surgical tissue guard shown in the accompanying drawings, which are for illustrative purposes only. These drawings include the following figures, in which like numerals indicate like parts:

FIG. 1 is a front perspective view of a typical forceps and a preferred embodiment of the present surgical tissue guard;

FIG. 2 is a front perspective view of the forceps and surgical tissue guard of FIG. 1, illustrating the manner in which the guard attaches to the forceps;

FIG. 3 is a top plan view of the surgical tissue guard of FIG. 1;

FIG. 4 is a front elevational view of the surgical tissue guard of FIG. 1;

FIG. 5 is a left-side elevational view of the surgical tissue guard of FIG. 1;

FIG. 6 is a left-side elevational view of the forceps and surgical tissue guard of FIG. 1, illustrating a first position of the guard upon the forceps;

FIG. 7 is a left-side elevational view of the forceps and surgical tissue guard of FIG. 1, illustrating a second position of the guard upon the forceps;

FIG. 8 is a front perspective view of another preferred embodiment of the present surgical tissue guard;

FIG. 9 is a left-side elevational view of the surgical tissue guard of FIG. 8 mounted upon the forceps of FIG. 1, illustrating a hinged closure of the guard in an open position;

FIG. 10 is a left-side elevational view of the surgical tissue guard of FIG. 8 mounted upon the forceps of FIG. 1, illustrating a hinged closure of the guard in a closed position;

FIG. 11 is a front elevational view of the surgical tissue guard of FIG. 8 mounted upon the forceps of FIG. 1, illustrating a hinged closure of the guard in an open position;

FIG. 12 is a front elevational view of the surgical tissue guard of FIG. 8 mounted upon the forceps of FIG. 1, illustrating a hinged closure of the guard in a closed position;

FIG. 13 is a front perspective view of another preferred embodiment of the present surgical tissue guard mounted upon the forceps of FIG. 1;

FIG. 14 is a top plan view of the surgical tissue guard of FIG. 13;

FIG. 15 is a front elevational view of the surgical tissue guard of FIG. 13;

FIG. 16 is a left-side elevational view of the surgical tissue guard of FIG. 13;

FIG. 17 is a front perspective view of another preferred embodiment of the present surgical tissue guard, wherein the guard is integral to a forceps;

FIG. 18 is a front perspective view of another preferred embodiment of the present surgical tissue guard;

FIG. 19 is a front elevational view of the surgical tissue guard of FIG. 18;

FIG. 20 is a right-side elevational view of the surgical tissue guard of FIG. 18;

FIG. 21 is a front perspective view of the surgical tissue guard of FIG. 18, illustrating the guard attached to the forceps;

FIG. 22 is a front perspective view of another preferred embodiment of the present surgical tissue guard;

FIG. 23 is a front perspective view of the surgical tissue guard of FIG. 22, illustrating the guard attached to the forceps;

FIG. 24 is a front perspective view of another preferred embodiment of the present surgical tissue guard;

FIG. 25 is a front elevational view of the surgical tissue guard of FIG. 24;

FIG. 26 is a top plan view of the surgical tissue guard of FIG. 24;

FIG. 27 is a front perspective view of the surgical tissue guard of FIG. 24, illustrating the guard attached to the forceps;

FIG. 28 is a front perspective view of another preferred embodiment of the present surgical tissue guard;

FIG. 29 is a front perspective view of the surgical tissue guard of FIG. 28, illustrating the guard attached to the forceps;

FIG. 30 is a rear perspective view of another preferred embodiment of the present surgical tissue guard, illustrating rotatable clips of the guard in a first orientation relative to a barrier portion of the guard;

FIG. 31 is a rear perspective view of the surgical tissue guard of FIG. 30, illustrating rotatable clips of the guard in a second orientation relative to the barrier portion of the guard;

FIG. 32 is a rear perspective view of the surgical tissue guard of FIG. 30, illustrating rotatable clips of the guard in a third orientation relative to the barrier portion of the guard;

FIG. 33 is a front perspective view of the surgical tissue guard of FIG. 30, illustrating the guard attached to the forceps;

FIG. 34 is a front perspective view of the surgical tissue guard of FIG. 30, illustrating the guard attached to the forceps;

FIG. 35 is a front perspective view of the surgical tissue guard of FIG. 30, illustrating the guard attached to the forceps;

FIG. 36 is a rear perspective view of another preferred embodiment of the present surgical tissue guard, illustrating rotatable clips of the guard in a first orientation relative to a barrier portion of the guard;

FIG. 37 is a rear perspective view of the surgical tissue guard of FIG. 36, illustrating rotatable clips of the guard in a second orientation relative to the barrier portion of the guard;

FIG. 38 is a rear perspective view of the surgical tissue guard of FIG. 36, illustrating rotatable clips of the guard in a third orientation relative to the barrier portion of the guard;

FIG. 39 is a front perspective view of the surgical tissue guard of FIG. 36, illustrating the guard attached to the forceps;

FIG. 40 is a front perspective view of the surgical tissue guard of FIG. 36, illustrating the guard attached to the forceps;

FIG. 41 is a front perspective view of the surgical tissue guard of FIG. 36, illustrating the guard attached to the forceps;

FIG. 42 is a rear perspective view of another preferred embodiment of the present surgical tissue guard, illustrating rotatable clips of the guard in a first orientation relative to a barrier portion of the guard;

FIG. 43 is a rear perspective view of the surgical tissue guard of FIG. 42, illustrating rotatable clips of the guard in a second orientation relative to the barrier portion of the guard; and

FIG. 44 is a front perspective view of the surgical tissue guard of FIG. 42.

### DETAILED DESCRIPTION OF THE PREFERRED EMBODIMENTS

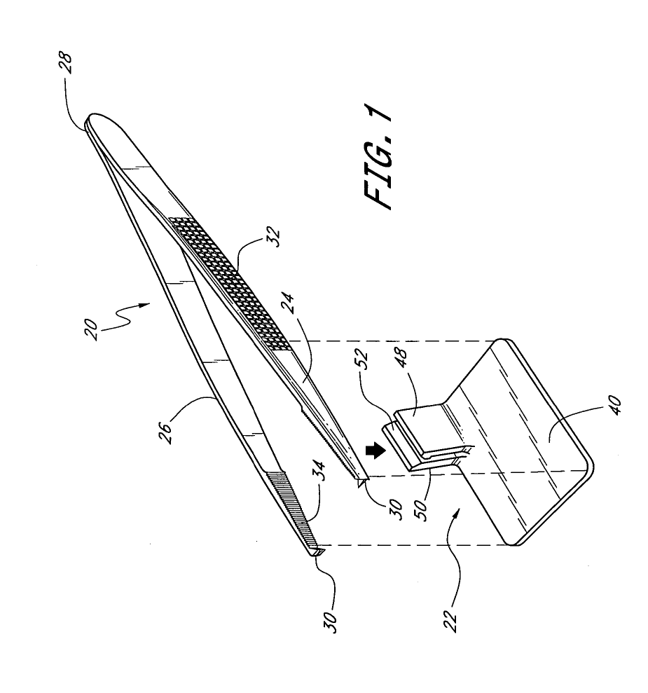

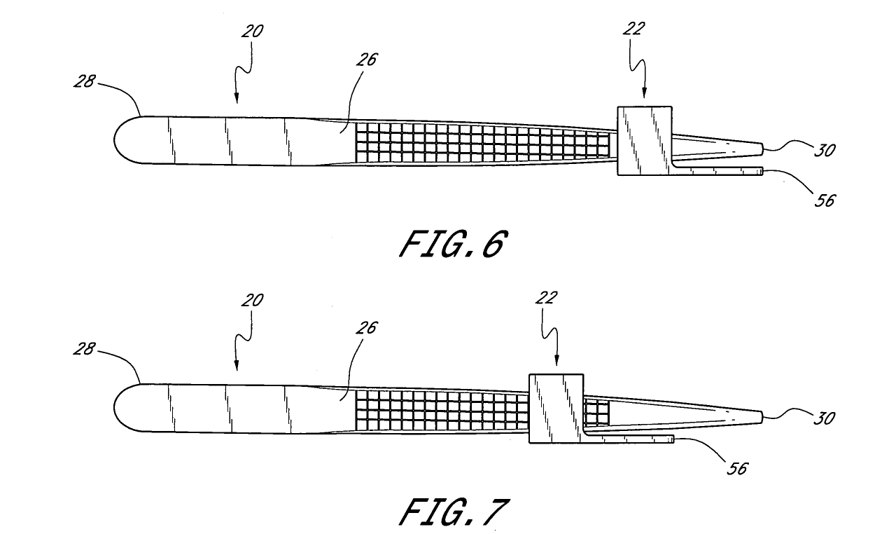

FIG. 1 illustrates a typical forceps 20 and a preferred embodiment of the present surgical tissue guard 22. Surgeons use the forceps 20 to manipulate bodily tissues during surgical procedures. The forceps 20 comprises a first arm 24 and a second arm 26 hingedly secured to one another at a first end 28. In side elevational aspect (FIGS. 6 and 7), a height of each arm 24, 26 tapers, becoming progressively shorter from the first end 28 toward a second end 30. The forceps 20 are typically constructed of metal, such as titanium or stainless steel.

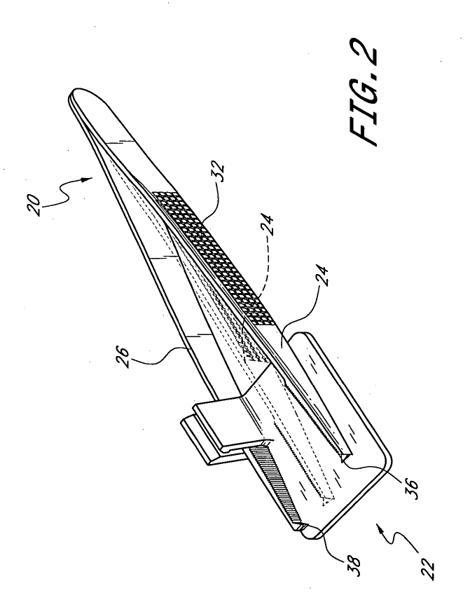

With reference to FIG. 1, an outer surface of each arm 24, 26 includes, near a center thereof, a textured portion 32. The textured portion 32 comprises a plurality of square bosses arranged in a grid pattern. The bosses provide a more easily grippable surface. A surgeon can grip the forceps 20 by positioning his or her thumb on the textured portion 32 of the first arm 24, and one or more fingers on the textured portion 32 of the second arm 26. By squeezing his or her thumb and fingers together, the surgeon brings the forceps' arms 24, 26 together, as illustrated in phantom in FIG. 2. After positioning the arms 24, 26 on either side of some tissue, the surgeon grasps the tissue by squeezing the arms 24, 26 together.

Inner surfaces of the arms 24, 26 include ribs 34 adjacent the second end 30 of each. The ribs 34 are substantially perpendicular to a longitudinal axis of the forceps 20. The ribs 34 provide increased grip, so that tissue captured between the arms 24, 26 does not slip free of the arms 24, 26.

To further increase the gripping ability of the forceps 20, the inner surface of the first arm 24 includes, at the second end thereof, a sharply-pointed prong 36 (FIG. 2) extending toward the second arm 26. The second arm 26 includes, opposite the first arm prong 36, a pair of prongs 38 spaced from one another in a direction parallel to the ribs 34. As the first arm 24 approaches the second arm 26 (phantom lining in FIG. 2), the first arm prong 36 enters the space between the second arm prongs 38. The prongs 36, 38 cooperate to hold firmly any tissue located between the second ends 30 of the first and second arms 24, 26. The prongs 36, 38 are sharp enough to puncture certain soft tissues, further enhancing the forceps' 20 gripping ability.

The forceps 20 described above are merely illustrative of one surgical instrument that is adapted for use with the preferred embodiments of the present surgical tissue guard. Those of skill in the art will appreciate that the preferred embodiments of the present surgical tissue guard are adapted for use with a variety of different forceps and other surgical instruments. These different forceps and other surgical instruments may or may not embody all of the structural details of the forceps 20 described above.

FIG. 1 also illustrates a preferred embodiment of the present surgical tissue guard 22. In the illustrated embodiment, the guard 22 comprises a barrier portion 40 shaped as a substantially rectangular flat plate. Those of skill in the art will appreciate that the barrier portion 40 could have a variety of other shapes, such as a concave/convex plate, a flat plate having more or fewer than four edges, or a flat plate having one or more straight edges and one or more curved edges.

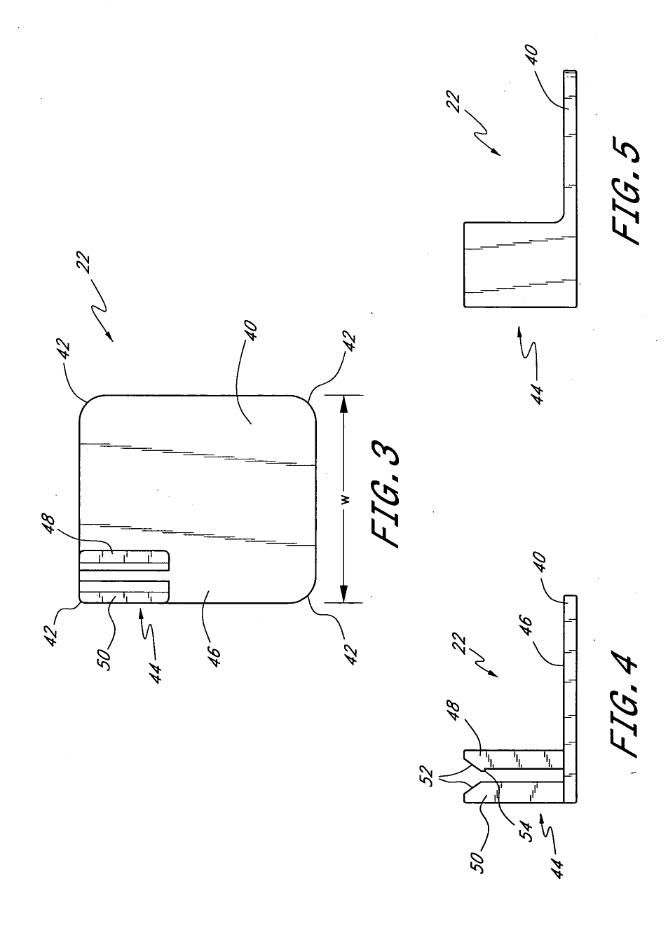

The barrier portion 40 preferably has a width W (FIG. 3) that is slightly. larger than a distance between the second ends 30 of the forceps' arms 24, 26 when the arms 24, 26 are in their relaxed configuration (solid lines in FIG. 2). Thus, when the guard 22 is secured to the forceps 20, as illustrated in FIG. 2, the guard 22 provides a complete barrier at the second ends 30 of the forceps' arms 24, 26. The guard 22 prevents tissue from getting between the second ends 30 of the forceps' arms 24, 26 from beneath the forceps 20.

In the illustrated embodiment, corners 42 (FIG. 3) of the barrier portion 40 are preferably rounded so that the corners 42 do not jab or irritate surrounding tissue as a physician manipulates tissue using the forceps 20 with the guard 22 attached. Those of skill in the art will appreciate that the corners 42 need not be rounded.

Referring to FIGS. 3-5, an attachment portion 44 extends substantially perpendicularly from a top surface 46 of the barrier portion 40 near a rear-left corner 42 thereof. The attachment portion 44 comprises a clip having a first side wall 48 and second side wall 50 opposing the first side wall 48. Preferably, an inner edge 52 (FIG. 4) of the front surface of each side wall 48, 50 slopes inwardly, i.e. toward the opposite side wall. (With reference to the side walls of the various clips disclosed herein, the term “inner” refers to the space between the side walls.) Thus, when the forceps' second arm 26 is positioned just above and between the side walls 48, 50, and then moved downward between the side walls 48, 50 (in the direction of the arrow shown in FIG. 1), the sloped surfaces 52 guide the second arm 26 into the space between the side walls 48, 50.

The first side wall 48 preferably includes a barb 54 (FIG. 4) on the inner surface thereof just below the sloped surface 52. The barb 54 comprises a protrusion from the first side wall 48, with surfaces of the barb 54 and the first side wall 48 intersecting at a ninety-degree angle. A distance between the side walls 48, 50 below the barb is preferably substantially equal to the thickness of the forceps' arms 24, 26 (as measured in the direction of the width W of the barrier portion 40, as in FIG. 3). Thus, the barb 54 protrudes above the forceps' second arm 26, maintaining the arm 26 between the side walls 48, 50. To withdraw the second arm 26 from between the side walls 48, 50 (in the direction opposite the arrow shown in FIG. 1), the side walls 48, 50 must flex to remove the barb 54 from the withdrawal path of the second arm 26.

Those of skill in the art will appreciate that the barb 54 could be located on the second side wall 50 instead of on the first side wall 48. Alternatively, both side walls 48, 50 could include barbs 54. Those of skill in the art will further appreciate that the attachment portion 44 could be secured to the forceps' first arm 24.

A perpendicular distance between the top surface 46 of the barrier portion 40 and the barb 54 is preferably equal to a depth of the forceps' arms 24, 26 near a midpoint thereof. The guard 22 is thus slidable along a length of the second arm 26 from a first position shown in FIG. 6 to a second position shown in FIG. 7. In the first position, a front edge 56 of the guard 22 is preferably substantially aligned with the second ends 30 of the forceps' arms 24, 26. In the second position, the second ends 30 of the forceps' arms 24, 26 protrude substantially from the front edge 56 of the guard 22. Thus, the guard 22 is readily slidable along the forceps 20 from a position in which the guard 22 prevents tissue from getting between the second ends 30 of the forceps' arms 24, 26 to a position in which the guard 22 does not prevent tissue from getting between the second ends 30. A physician using a forceps 20 with the guard 22 attached can easily slide the guard 22 out of the way in order to use the forceps 20 as he or she normally would without the guard 22 attached.

The guard 22 is preferably constructed of a bio-compatible, rigid, puncture resistant material. However, the guard may be constructed of a semi-rigid material, or a combination of rigid and semi-rigid materials. Such semi-rigid materials are preferably able to flex a small amount. The material is preferably substantially transparent, so that a physician can see through the guard 22 to the tissue that he or she is displacing or protecting with the guard 22. However, the guard may be constructed of an opaque material, or a combination of transparent and opaque materials. The material may be radio-transparent, or radio-opaque/radio-lucent, or a combination thereof. If the material is radio-opaque/radio-lucent, the guard will advantageously be visible from outside the patient's body. The guard can thus be easily recovered in case it is accidentally left inside the patient. The material should be non-toxic and safe for use internally. In order to facilitate easy attachment to and detachment from the forceps 20, the material is preferably flexible enough to allow the side walls 48, 50 to flex as the forceps' second arm 26 is inserted into the space between the side walls 48, 50. Preferred materials include bio-compatible plastics, and polycarbonates, such as Lexan®, and metals such as titanium and stainless steel.

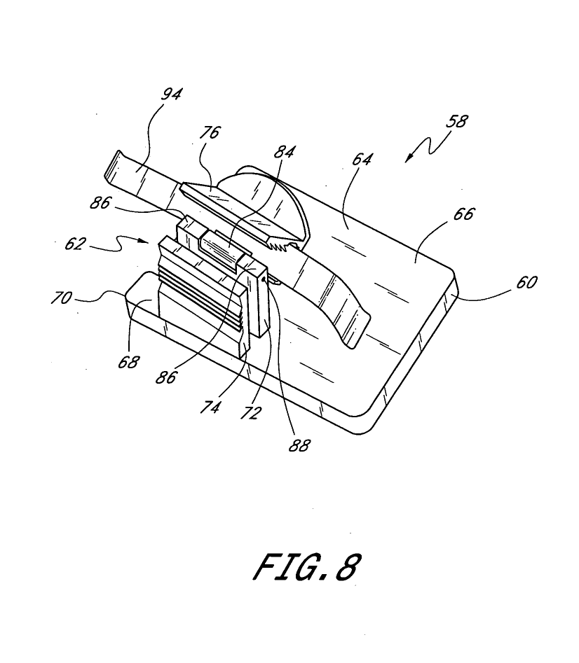

FIGS. 8-12 illustrate another preferred embodiment of the present surgical tissue guard 58. Similar to the guard 22 illustrated in FIGS. 1-7, the guard 58 comprises a barrier portion 60 with an attachment portion 62 protruding from a top surface 64 thereof (FIG. 8).

In the illustrated embodiment, the barrier portion 60 includes a substantially flat and rectangular main body portion 66, and a coplanar, substantially rectangular “panhandle” 68 attached at an upper left-hand corner 70. The attachment portion 62 may be attached, wholly or partially, to the panhandle 68. Those of skill in the art will appreciate that the barrier portion 60 could embody virtually any shape, and need not include a panhandle 68. Advantageously, however, the panhandle 68 provides additional surface area on the barrier portion 60 to which the attachment portion 62 can be secured.

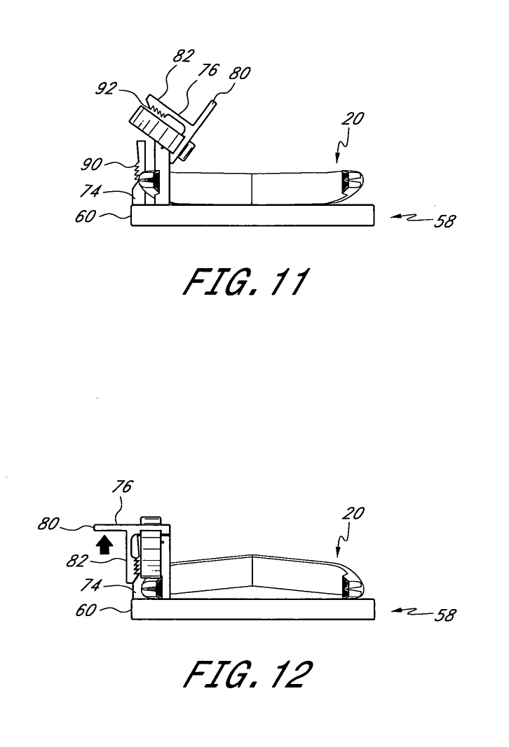

The attachment portion 62 comprises a first side wall 72 and a second side wall 74 substantially parallel to the first side wall 72 (FIG. 8). A cover 76 is hingedly secured to a front end 78 of the first side wall 72. As illustrated in FIGS. 11 and 12, the cover 76 comprises a first planar portion 80 and a second planar portion 82 extending substantially perpendicularly from a lower surface of the first planar portion 80, such that the cover 76 resembles a T in front elevational aspect (FIG. 12). With reference to FIG. 8, a first hinge portion 84 extends downward from the lower surface of the first planar portion 80 along a right edge thereof. The first hinge portion 84 seats between second hinge portions 86 at the upper end of the first side wall 72 (FIG. 8). A hinge pin 88 extends through the first and second hinge portions 84, 86 to pivotably secure the cover 76 to the first side wall 80.

The cover 76 is pivotable between an open position (FIG. 11) and a closed position (FIG. 12). In the open position, the cover 76 does not obstruct the opening at the upper ends of the side walls 72, 74, enabling the forceps' second arm 26 to be inserted into and removed from the space between the side walls 72, 74. In the closed position, the cover 76 blocks the opening at the upper ends of the side walls 72, 74, locking the forceps' second arm 26 between the side walls 72, 74, or preventing the second arm 26 from being inserted between the side walls 72, 74.

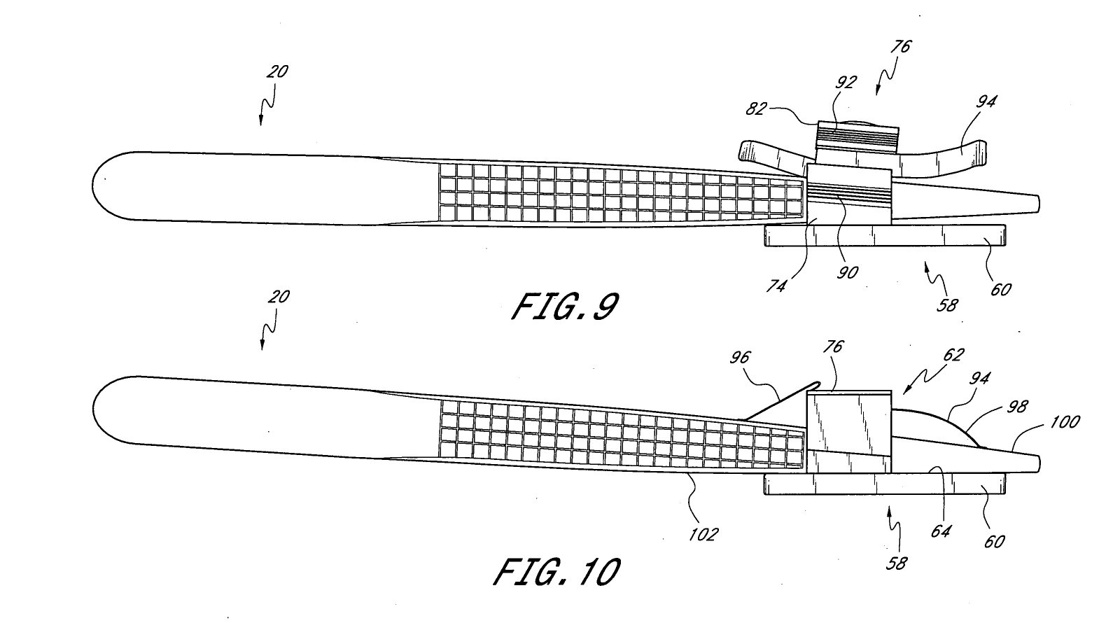

When the cover 76 is in the closed position (FIG. 12) an inner surface of the second planar portion 82 contacts an outer surface of the second side wall 74. The outer surface of the second side wall 74 includes ratchet teeth 90 (FIGS. 9 and 11). The inner surface of the second planar portion 82 includes complementary ratchet teeth 92. Thus, when the cover 76 is in the closed position, the ratchet teeth 92 on the second planar portion 82 engage the ratchet teeth 90 on the second side wall 74 to secure the cover 76 in the closed position. To pivot the cover 76 from the closed position to the open position, a user applies an upward force to the overhanging portion of the first planar portion 80, as shown by the arrow in FIG. 12. The force causes the first planar portion 80 to flex, which causes the ratchet teeth 92 on the second planar portion 82 to disengage the ratchet teeth 90 on the second side wall 74, thus releasing the cover 76.

A leaf spring 94 (FIGS. 8-10) is secured to the lower surface of the first planar portion 80. In the illustrated embodiment, the leaf spring 94 comprises a thin metallic strip. When the cover 76 is in the closed position, as illustrated in FIG. 10, a rear portion 96 of the leaf spring 94 extends in a substantially straight line downward and rearward from the rear edge of the attachment portion 62. A front portion 98 of the leaf spring 94 extends in an arc downward and forward from the front edge of the attachment portion 62. Opposite ends of the leaf spring 94, which are bent forward slightly, contact an upper edge 100 of the forceps second arm 26, as shown in FIG. 10. The leaf spring 94 pushes downward on the front edge 100, which brings the top surface 64 of the barrier portion 60 into continuous contact with a lower edge 102 of the forceps' second arm 26 (FIG. 10). The leaf spring 94 thus enables the guard 58 to maintain a firm hold on the forceps' second arm 26, and prevents the guard 58 from accidentally becoming detached from the forceps' second arm 26.

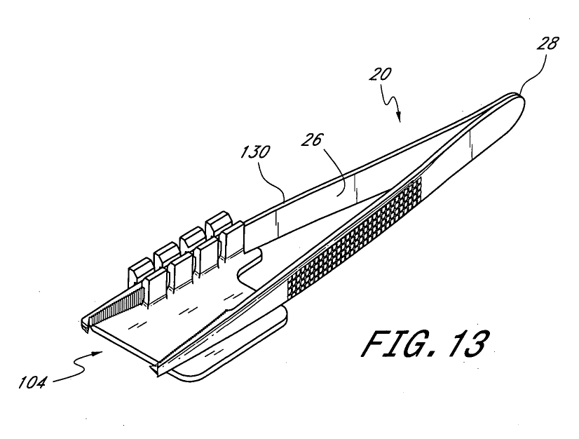

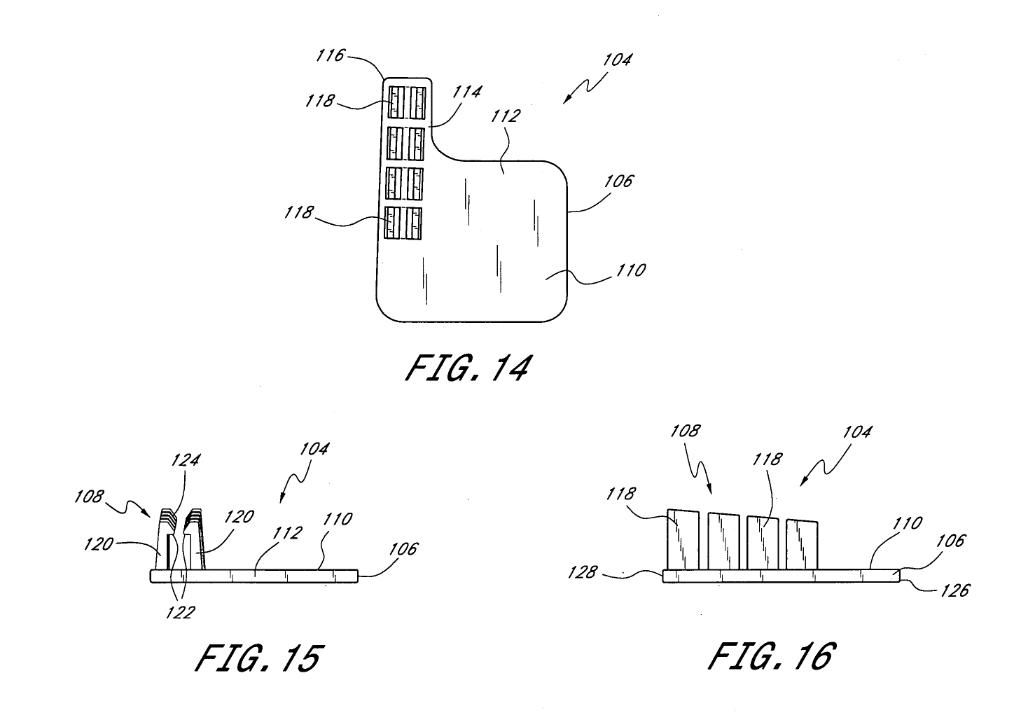

FIGS. 13-16 illustrate another preferred embodiment of the present surgical tissue guard 104. With reference to FIGS. 14-16, the guard 104 comprises a barrier portion 106 with an attachment portion 108 extending from a top surface 110 thereof. In the illustrated embodiment, the barrier portion 106 is shaped as a flat plate having a substantially rectangular main body portion 112, and a substantially rectangular panhandle portion 114 attached to a rear-left corner 116 (FIG. 14). Those of skill in the art will appreciate that the barrier portion 106 could be any desired shape or dimension.

The attachment portion 108 comprises a plurality of clips 118. In the illustrated embodiment, four clips 118 are provided. Two of the clips 118 are attached to the main body portion 112, one of the clips 118 is attached to the panhandle portion 114, and one of the clips 118 is partially attached to both portions 112, 114. Those of skill in the art will appreciate that more or fewer clips 118 could be provided. Those of skill in the art will also appreciate that the barrier portion 106 need not include a panhandle portion 114, and that the clips 118 could be distributed differently between the main body portion 112, and the panhandle portion 114.

Each clip 118 comprises substantially parallel side walls 120 (FIG. 15). Each side wall 120 includes a barb 122. The barbs 122 each comprise a protrusion from their respective side wall 120. Like the barb 54 described above with reference to the guard 22 of FIGS. 1-7, the barbs 122 form an obstruction that resists the withdrawal of the forceps' second arm 26 from the space between the side walls 120. Those of skill in the art will appreciate that the barbs 122 need not be provided on every side wall 120.

An upper inner surface 124 (FIG. 15) of each side wall 120 is sloped to guide the forceps' second arm 26 into the space between the side walls 120. A length of each clip 118, as measured in a direction from the front edge 126 to the rear edge 128 of the guard 104 (FIG. 16), is substantially shorter than a length of the attachment portion 44 described above with reference to the guard 22 of FIGS. 1-7. Thus, the side walls 120 of each clip 118 are more flexible than the side walls 48, 50 of the clip 44 of FIGS. 1-7. The forceps' second arm 26 thus slides more easily into and out of the space between the side walls 120.

A thickness (FIG. 15) of each side wall 120 is preferably selected, based upon the material properties of the clips 118, to provide the side walls 120 with sufficient strength to firmly grip the forceps' second arm 26. In the illustrated embodiment, a thickness of each side wall 120 decreases with increasing distance from the barrier portion 106. The flexibility of each side wall 120 thus increases with increasing distance from the barrier portion 106. Those of skill in the art will appreciate that the side walls 120 need not have variable thickness.

The clips 118 are slidable for a short distance along the length of the forceps' second arm 26 (FIG. 13). When the clips 118 reach the point where the height of the forceps' second arm 26 is equal to the distance between the barrier portion 106 and the barbs 122, the guard 104 cannot slide any farther toward the forceps' first end 28. A height of each clip 118, as measured in a direction perpendicular to the barrier portion 106 (FIG. 16), increases from the front end 126 of the barrier portion 106 to the rear end 128 thereof. The slope of the increasing heights preferably tracks the slope of the forceps' second arm 26.

Similarly, a distance between the barrier portion 106 and the barb 122 of each clip 118 increases from the front end 126 of the barrier portion 106 to the rear end 128 thereof. The surfaces of each barb that contact the forceps 20 when the guard 104 is attached to the forceps 20, as shown in FIG. 13, are preferably sloped at an angle corresponding to the taper angle of the forceps' second arm 26. Thus, when the guard 104 is positioned on the forceps 20 such that it is as close to the first end 28 as possible, each barb 122 maintains continuous contact with the upper edge 130 of the second arm 26. Furthermore, a total length of the four clips 118 combined is greater than the length of the clip 44 of FIGS. 1-7. The clips 118 thus grip the forceps' second arm 26 in such a manner as to prevent the guard 104 from rotating relative to the forceps 20 about an axis passing through both forceps' arms 24, 26.

The embodiments of the guard 22, 58, 104 described above are easily detachable from the forceps 20. Thus, the guards 22, 58, 104 are advantageously disposable. After one of the guards 22, 58, 104 is used in a surgical procedure, the surgeon or hospital need not clean and sterilize the guard 22, 58, 104. The surgeon or hospital simply discards the guard 22, 58, 104. For the next surgical procedure, the surgeon uses a new guard 22, 58, 104. Disposing of the old guard 22, 58, 104 and using a new one for each surgical procedure reduces the chances of contaminants being transferred from one patient to another. Of course, if desired, the guards 22, 58, 104 may be reused. If they are to be reused, preferably the guards 22, 58, 104 are constructed of a material that is durable enough to withstand repeated uses and the necessary washing and sterilizing between each use.

Those of skill in the art will appreciate that the present surgical tissue guard could be permanently attached to the forceps. For example, the tissue guard could be formed integrally with the forceps. Alternatively, the tissue guard could be formed separately from a typical forceps and then permanently attached to the forceps.

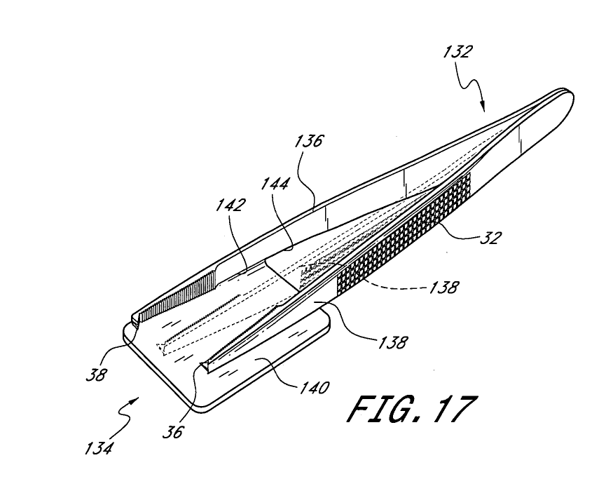

FIG. 17 illustrates a forceps 132 in combination with a tissue guard 134, wherein the tissue guard 134 is not readily detachable from the forceps 132. The forceps 132 pictured in FIG. 17 is substantially identical to the forceps 20 described above, except for the addition of the tissue guard 134. In the illustrated embodiment, the guard 134 is permanently attached to a first arm 136 of the forceps, such that a second arm 138 is movable with respect to the guard 134. However, those of skill in the art will appreciate that the guard 134 could be permanently attached to the second arm 138, such that the first arm 136 is movable with respect to the guard 134.

The guard 134 comprises a barrier portion 140 that is shaped as a substantially flat rectangular plate. Of course, the barrier portion 140 could embody a variety of other shapes, such as a concave plate, or a flat plate having one or more curved edges. An edge 142 of the barrier portion 140 is permanently secured to a lower edge 144 of the first arm 136. The permanent attachment may comprise a weld. For example, if the forceps 132 and guard 134 are constructed of plastic, the guard 134 could be permanently attached to the forceps 132 with an ultrasonic weld. Alternatively, the guard 134 could be permanently attached to the forceps 132 with an adhesive. Alternatively, the guard 134 could be formed integrally with the forceps 132. For example, the forceps 132 and guard 134 could be molded from plastic, or cast from a metal.

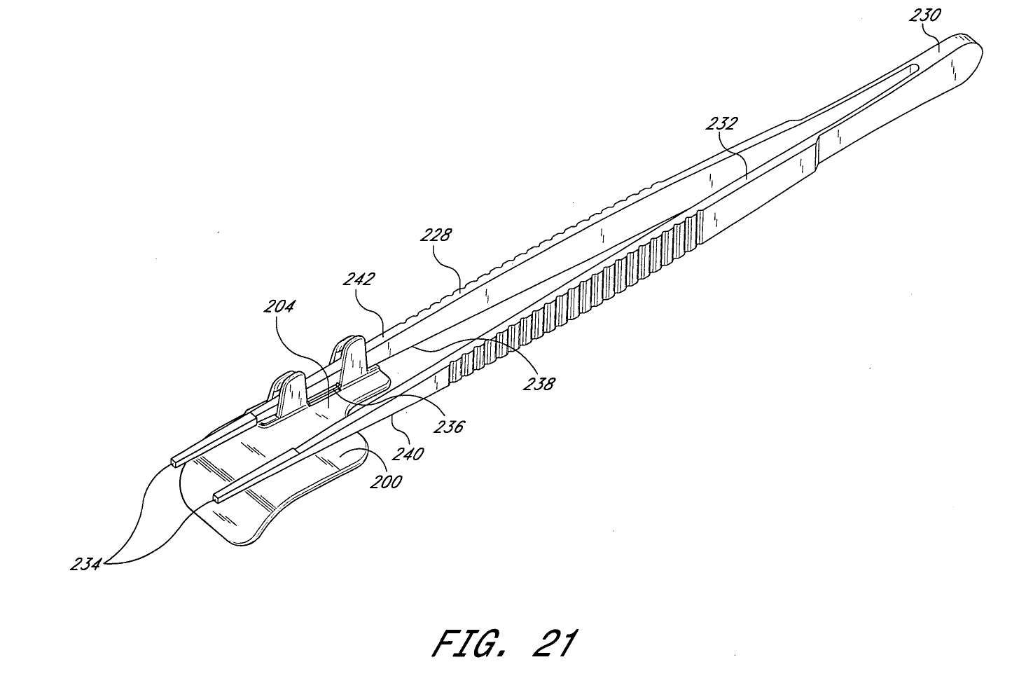

FIGS. 18-21 illustrate another preferred embodiment of the present surgical tissue guard. With reference to FIG. 18, the guard 200 includes a barrier portion 202 having a substantially flat region 204 and a curved region 206. The flat region 204 includes a panhandle portion 208 at a rear-left corner thereof. The curved region 206 extends forward and downward from a front portion 210 of the substantially flat region 204, such that in side elevation aspect the barrier portion 202 resembles a J (FIG. 20).

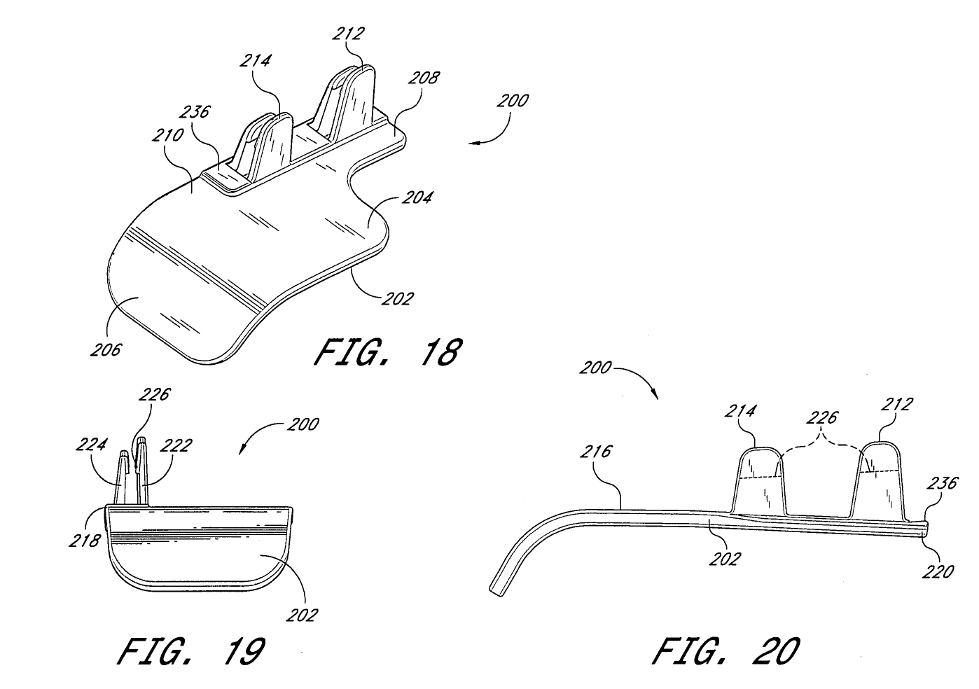

With reference to FIGS. 18 and 20, first and second clips 212, 214 extend substantially vertically from the upper surface 216 of the barrier portion 202. The clips 212, 214 are situated along a left edge 218 (FIG. 19) of the barrier portion 202 toward the rear 220 (FIG. 20) thereof. A rear clip 212 resides atop the panhandle portion 208, with a forward clip 214 situated in front of the rear clip 212. The rear clip 212 may be somewhat taller than the forward clip 214, as illustrated in FIG. 20.

Each clip 212, 214 comprises a first side wall 222 and a second side wall 224 (FIG. 19). Preferably, each side wall 222, 224 includes a barb 226 near an upper end of each. Those of skill in the art will appreciate that neither side wall 222, 224 need include a barb 226, and that a barb 226 may be provided on only one of the side walls 222, 224.

Preferably, a first side wall 222 of each clip 212, 214 is slightly taller than a second side wall 224 thereof. The taller side wall 222 is easily bent away from the shorter side wall 224 under digital pressure. The taller/shorter configuration thus enables a user to more easily separate the side walls 222, 224 in order to insert a forceps 230 into the clips 212, 214 or remove a forceps 230 from between the clips 212, 214. In the illustrated embodiment, the shorter side walls 224 are located along the left edge 218 of the guard 200. However, those of skill in the art will appreciate that the taller and shorter side walls could be reversed.

The guard 200 is releasably secureable to a first arm 228 of a forceps 230, as illustrated in FIG. 21. A second arm 232 of the forceps 230 is freely movable toward and away from the first arm 228 and across the barrier portion 202. The forceps 230 of FIG. 21 is similar to the forceps 20 described above, with a few minor differences. For example, the forceps 230 of FIG. 21 does not include ribs or prongs at second ends 234 of each arm 228, 232. Those of skill in the art will appreciate, however, that each of the tissue guards described herein is adapted for use with any of the forceps disclosed, and with a wide variety of other standard surgical forceps. The particular combinations of guards and forceps illustrated should not be interpreted as limiting.

With reference to FIGS. 18, 20 and 21, the clips 212, 214 preferably reside atop a raised strip 236 that extends along a rear portion of the left edge 218 of the barrier portion 202. The strip 236 provides a floor against which a lower edge 238 of the forceps' first arm 228 abuts when the guard 200 is secured to the forceps 230, as illustrated in FIG. 21. This configuration creates a small gap between a lower edge 240 of the forceps' second arm 232 and the flat region 204 of the barrier portion 202. This gap enables the second arm 232 to move toward the first arm 228 without interfering with a lower extent of the clips 212, 214.

When the guard 200 is manufactured using certain manufacturing methods, a transition from the barrier portion 202 to the clips 212, 214 is likely to include a rounded fillet. For example, if the guard 200 is molded from plastic, such a fillet is likely to exist at the transition. If there is no gap between the lower edge 240 of the second arm 232 and the barrier portion 202, then the lower edge 240 of the second arm 232 will contact the fillet before the second ends 234 of the first and second arms 228, 232 meet. The fillet will thus prevent the first and second arms 228, 232 from meeting. The spacing between the second arm 232 and the barrier portion 202 enables the first and second arms 228, 232 to meet. The raised strip 236 creates this spacing. Those of skill in the art will appreciate that the guard 200 need not include the raised strip 236.

With reference to FIG. 20, the barbs 226 at the upper ends of the clips 212, 214 preferably run diagonally with respect to the flat region 204 of the barrier portion 202. A rear end of each barb 226 is spaced a greater distance from the barrier portion 202 as compared to a front end of each barb 226. This configuration allows the barbs 226 to flushly engage the upper surface 242 of the first arm 228 (FIG. 21), which tapers down to a lesser height near the second end 234 thereof. Inner surfaces of each side wall 222, 224 may include substantially vertical ribs that aid the clips 212, 214 in gripping the first arm 228. Those of skill in the art will appreciate that the ribs need not be vertical.

The curved region 206 of the guard 200 provides a handy tool for tissue displacement. The curved region 206 forms a sort of a hook that is adapted to catch tissue and pull it in a first direction. The barrier portion 202 is preferably thick enough that the curved region 206 flexes only a small amount as it pulls tissue.

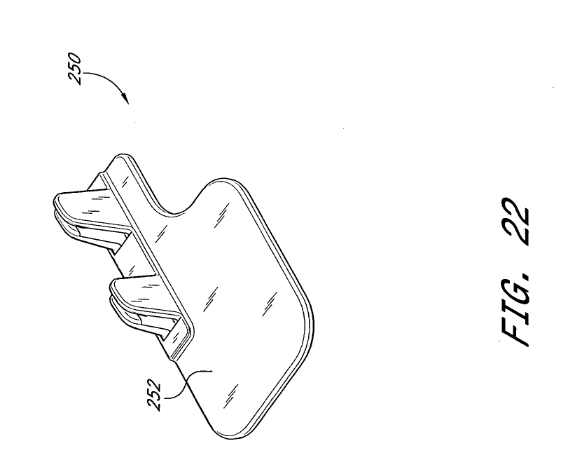

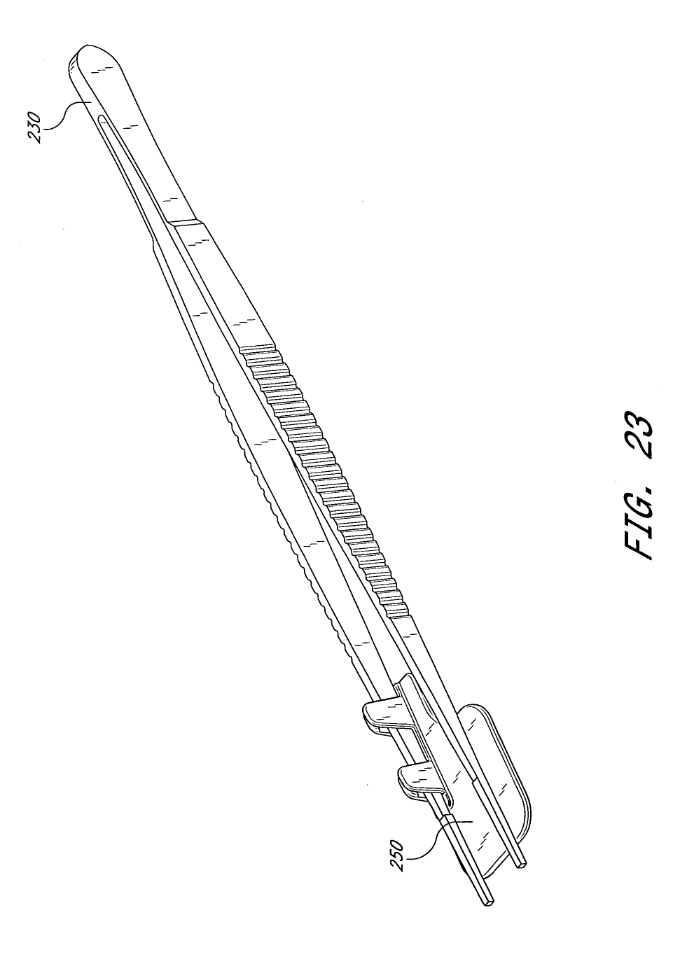

FIGS. 22 and 23 illustrate another preferred embodiment of the present surgical tissue guard. With reference to FIG. 22, the guard 250 is substantially identical to the guard 200 illustrated in FIGS. 18-21. However, the guard 250 of FIG. 22 includes a substantially flat barrier portion 252 with no curved portion. FIG. 23 illustrates the guard 250 secured to the forceps 230.

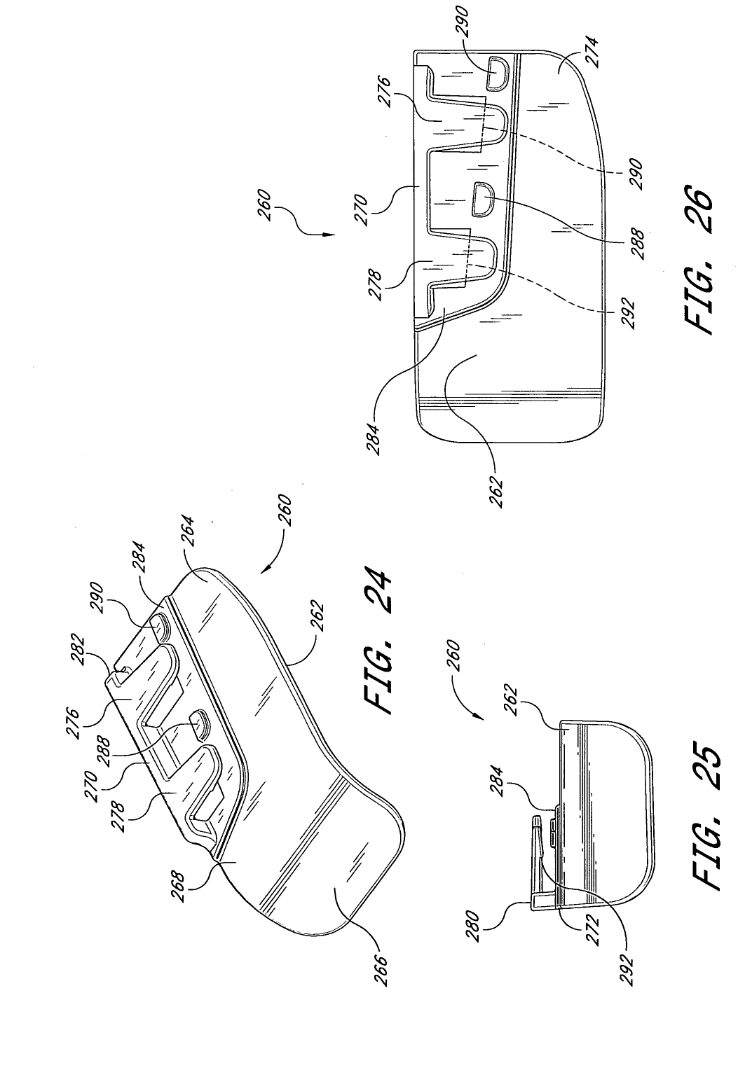

FIGS. 24-27 illustrate another preferred embodiment of the present surgical tissue guard. With reference to FIG. 24, the guard 260 includes a barrier portion 262 having a substantially flat region 264 and a curved region 266. The curved region 266 extends forward and downward from a front portion 268 of the substantially flat region 264, such that in side elevation aspect the barrier portion 262 resembles a J.

With reference to FIGS. 24 and 26, a short clip support wall 270 extends along a left edge 272 (FIG. 25) of the barrier portion 262 toward the rear 274 thereof. A first clip 276 and a second clip 278 extend substantially horizontally from an upper edge 280 of the clip support wall 270 (FIG. 25), such that the clips 276, 278 extend over the barrier portion 262. A rear clip 276 resides adjacent a rear end 282 of the wall 270, with a forward clip 278 situated in front of and spaced from the rear clip 276. The rear clip 276 may extend somewhat farther across the barrier portion 262 than the forward clip 278, as illustrated in FIG. 26.

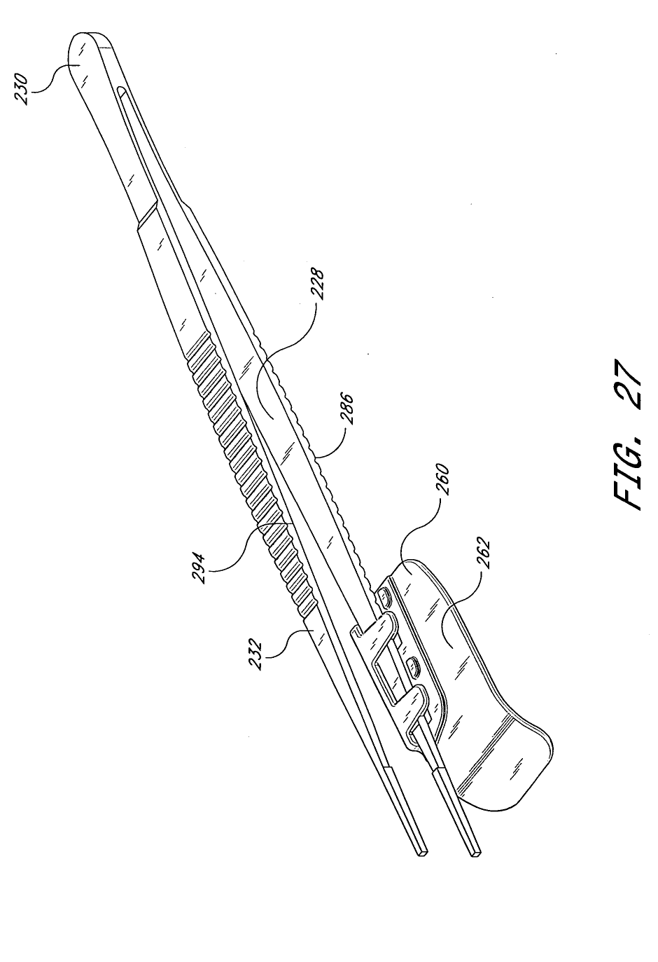

The guard 260 is releasably secureable to the first arm 228 of the forceps 230, as illustrated in FIG. 27. The second arm 232 of the forceps 230 is freely movable toward and away from the first arm 228 and toward and away from the barrier portion 262.

With reference to FIGS. 24-26, the clip support wall 270 preferably resides atop a raised strip 284 that extends along a rear portion of the left edge 272 of the barrier portion 262. The strip 284 provides a floor against which an outer surface 286 of the forceps' first arm 228 abuts when the guard 260 is secured to the forceps 230, as illustrated in FIG. 27. A forward boss 288 and a rear boss 290 (FIGS. 24 and 26) reside atop the raised strip 284, spaced from the clip support wall 270. The bosses 288, 290 aid in securing the guard 260 to the forceps 230, as described in detail below. The forward boss 288 is positioned between the forward and rear clips 276, 278 when the guard 260 is viewed in top plan aspect, as in FIG. 26. The rear boss 290 is positioned behind the rear clip 276 and adjacent to a rear 274 of the guard 260.

With reference to FIGS. 25 and 26, a lower surface of each clip 276, 278 includes a barb 292. The barbs 292 are situated near an end of each clip 276, 278 that is spaced from the support wall 270. The barbs 292 preferably run diagonally with respect to the clip support wall 270, as shown in FIG. 26. A rear end of each barb 292 is spaced a greater distance from the clip support wall 270 as compared to a front end of each barb 292. A surface of each boss 288, 290 that faces the clip support wall 270 includes a similar diagonal slope.

The surfaces of the barbs 292 and the surfaces of the bosses 288, 290 that face the clip support wall 270 preferably define a straight line. These surfaces are adapted to flushly engage a first surface 294 of the first arm 228, as illustrated in FIG. 27. Inner surfaces of each clip 276, 278 may include ribs (not shown) that aid the clips 276, 278 in gripping the first arm 228.

Referring to FIGS. 21 and 27, the guard 200 of FIG. 21 is adapted to be secured to the forceps 230 such that the guard 200 is disposed beneath each of the first and second arms 228, 232. By contrast, the guard 260 of FIG. 27 is adapted to be secured to the forceps 230 such that the guard 260 is disposed to the outside of the first arm 228. These configurations are thus adapted for use in a variety of different situations. Because the clips 276, 278 are readily removable and reattachable to the forceps 230, one guard 200, 260 can advantageously be switched for another having a different configuration. For example, a user may readily switch one guard 200, 260 for another during a surgical or suturing procedure.

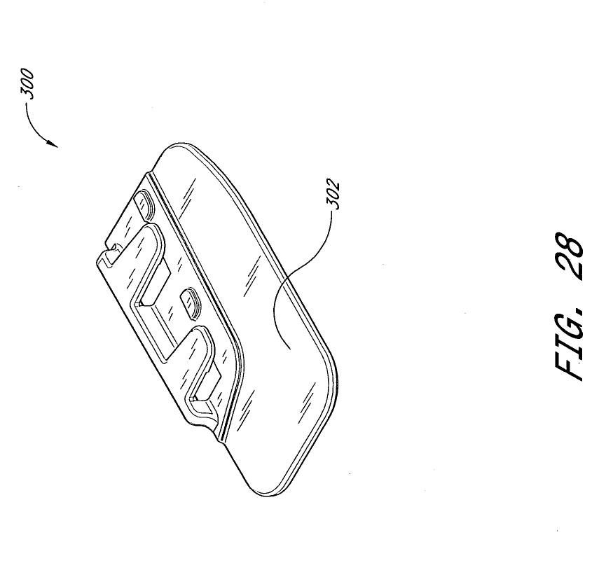

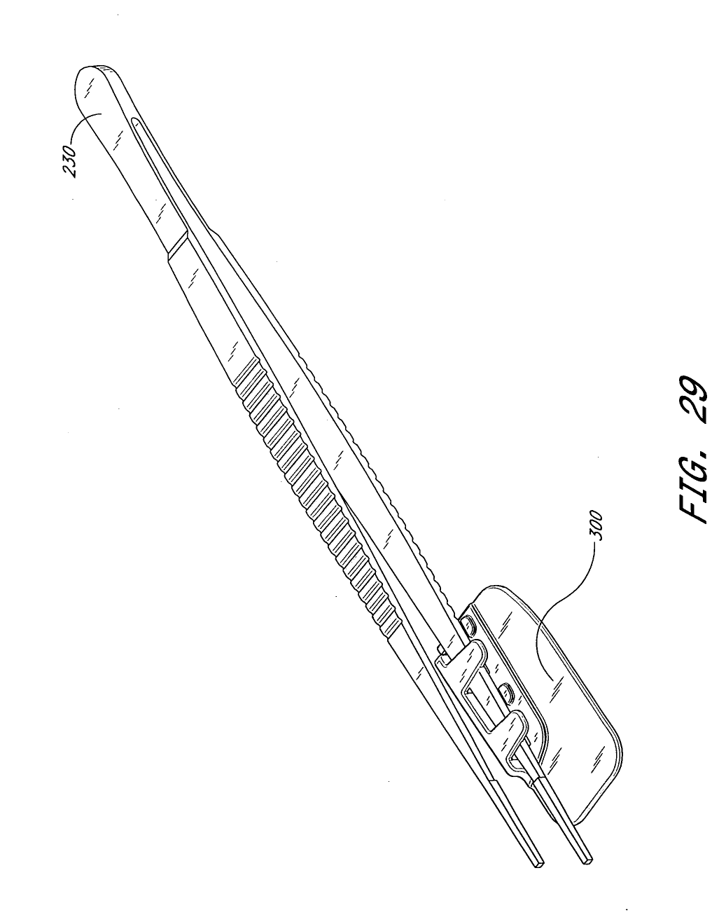

FIGS. 28 and 29 illustrate another preferred embodiment of the present surgical tissue guard. With reference to FIG. 28, the guard 300 is substantially identical to the guard 260 illustrated in FIGS. 24-27. However, the guard 300 of FIG. 28 includes a substantially flat barrier portion 302 with no curved portion. FIG. 29 illustrates the guard 300 secured to the forceps 230.

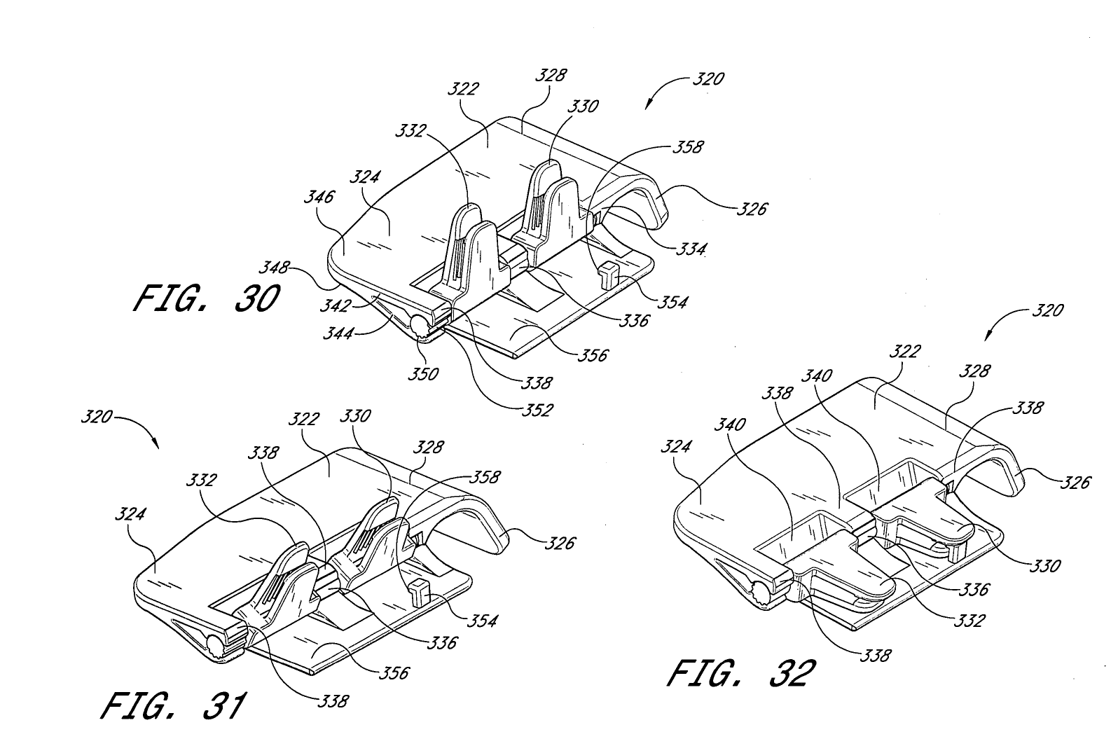

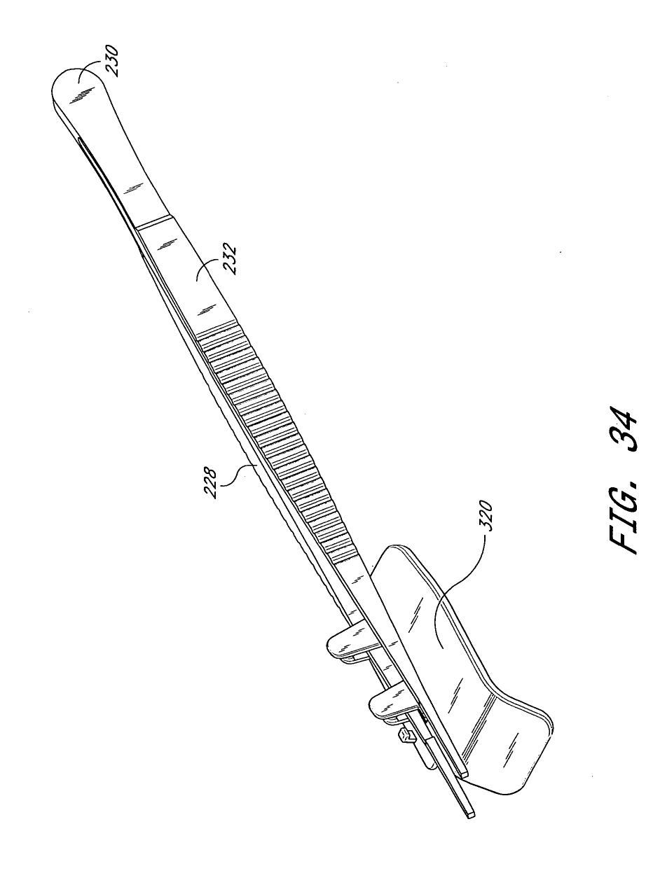

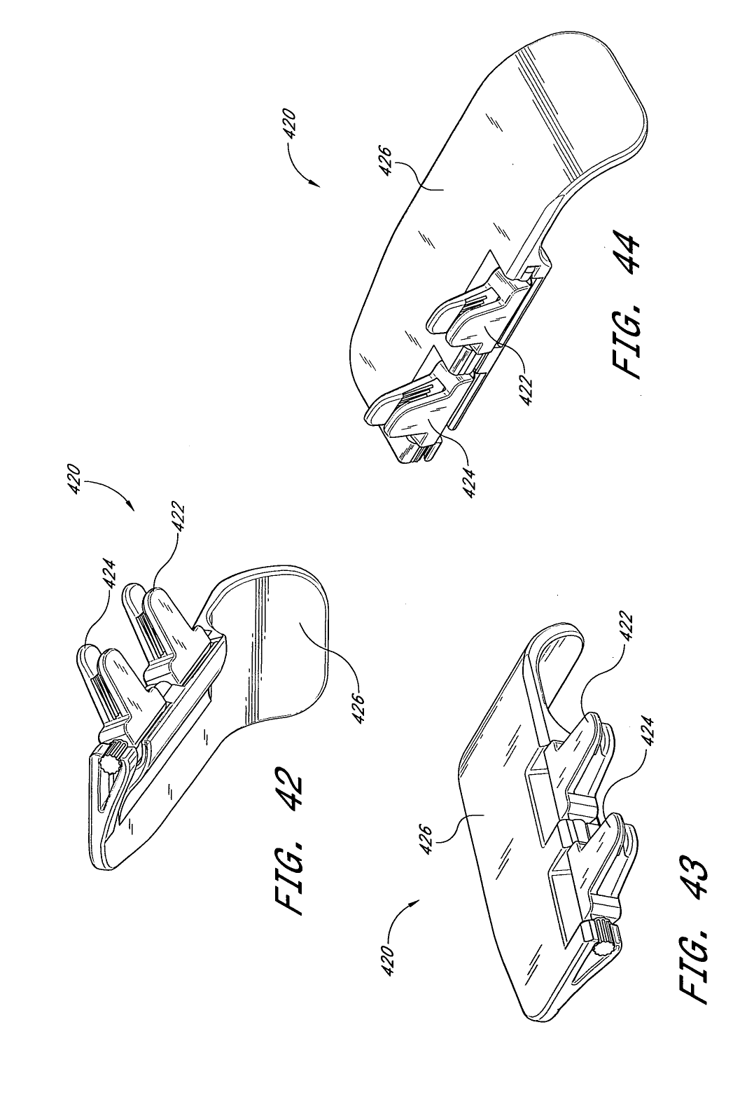

FIGS. 30-35 illustrate another preferred embodiment of the present surgical tissue guard 320 having pivotable clips 330, 332. FIGS. 42-44 illustrate a variation of the embodiment of FIGS. 30-35, wherein a locking mechanism is omitted. With reference to FIGS. 30-32, the guard 320 includes a barrier portion 322 having a substantially flat region 324 and a curved region 326. The curved region 326 extends forward and downward from a front portion 328 of the substantially flat region 324, such that in side elevational aspect the barrier portion 322 resembles a J.

A pair of clips 330, 332 are pivotably attached to the barrier portion 322 along a left edge 334 thereof (FIG. 30). With reference to FIG. 30, the clips 330, 332 are substantially identical to the clips 212, 214 illustrated in FIGS. 18-23. However, the clips 330, 332 illustrated in FIGS. 30-35 are secured at a lower end to a post 336. The post 336 extends between the two clips 330, 332, forward from the forward clip 330, and rearward from the rear clip 332. The exposed portions of the post 336 are captured within jaws 338 that extend from the barrier portion 322. The post 336 is rotatable within the jaws 338. FIGS. 30-32 illustrate examples of angular orientations of the clips 330, 332 relative to the barrier portion 322. Preferably, the barrier portion 322 includes first and second cut-out wells 340 (FIG. 32) that receive lower portions of the clips 330, 332.

With reference to FIG. 30, and using the rearmost set of jaws 338 as a model, each set of jaws 338 comprises an upper jaw 342 and a lower jaw 344. The upper jaw 342 is an extension of the upper surface 346 of the barrier portion 322, and extends toward the left side 334 of the guard 320. The lower jaw 344 similarly extends toward the left side 334 of the guard 320, but it extends diagonally downward from a lower surface 348 of the barrier portion 322. Facing surfaces at the ends of the upper and lower jaws 342, 344 include ratchet teeth 350. The ratchet teeth 350 mesh with ratchet teeth 352 on the post 336. The meshing ratchet teeth 350, 352 maintain the angular position of the clips 330, 332 relative to the barrier portion 322.

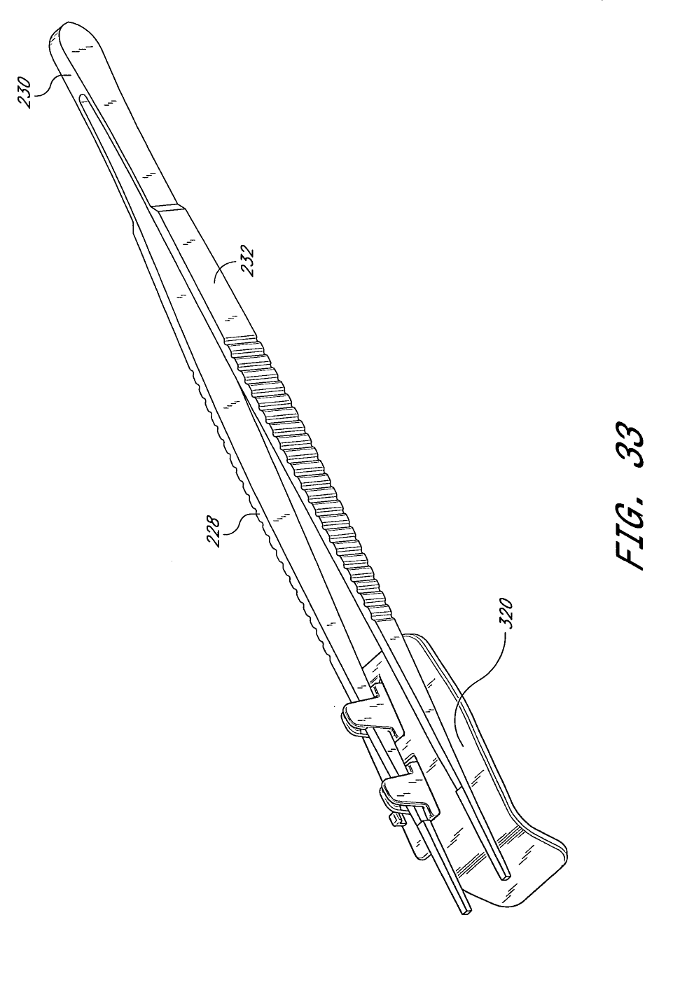

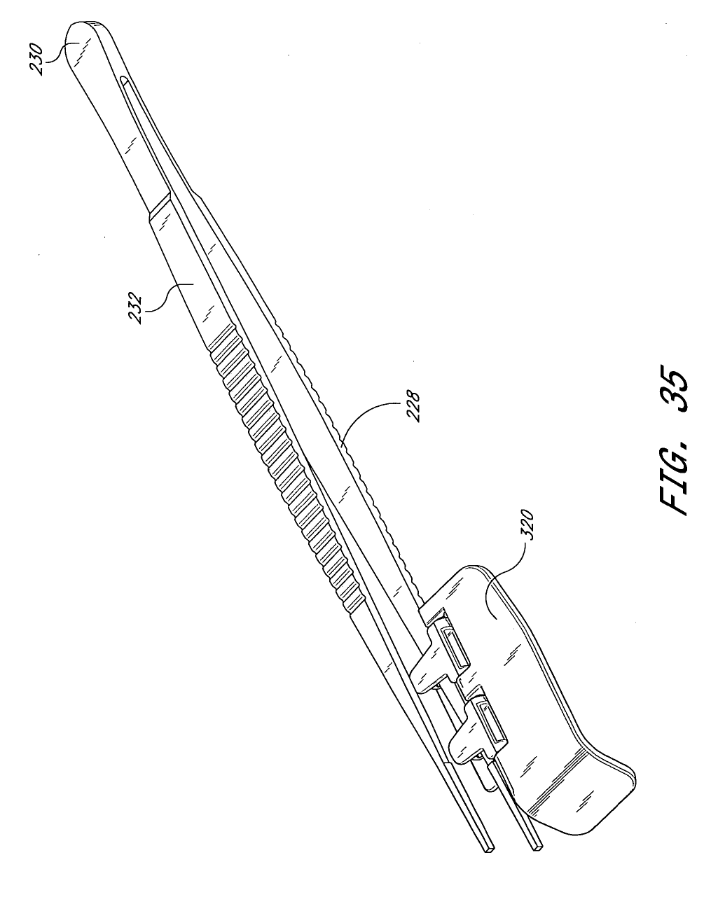

With reference to FIGS. 33-35, the guard 320 is releasably secureable to the first arm 228 of the forceps 230. A second arm 232 of the forceps 230 is freely movable toward and away from the first arm 228. The clips 330, 332 grip the first arm 228 in the same fashion as the clips 212, 214 described above with respect to the guards 200, 250 of FIGS. 18-23. The barrier portion 322 is pivotable about the first arm 228 to alter an orientation of the guard 320 relative to the forceps 230. Preferably, the guard 320 is pivotable about ninety-degrees between the orientations of FIGS. 33 and 35. In the orientation of FIG. 33, the barrier portion 322 is positioned beneath both arms 228, 232 of the forceps 230, and the second arm 232 is movable across the barrier portion 322. In this configuration, the barrier portion 322 blocks tissue positioned beneath the forceps 230. In the orientation of FIG. 35, the barrier portion 322 is positioned beneath and to the outside of the first arm 228, and the second arm 232 is movable toward the barrier portion 322. In this configuration, the barrier portion 322 blocks tissue positioned to the outside of the first arm 228. Those of skill in the art will appreciate that the guard 320 may be pivotable about the forceps 230 between angles of less than or more than ninety-degrees.

To twist the clips 330, 332 relative to the barrier portion 322, a user applies digital pressure to both the clips 330, 332 and the barrier portion 322. The user may twist the clips 330, 332 prior to securing the guard 320 on the forceps 230, or the user may twist the clips 330, 332 after securing the guard 320 on the forceps 230. If the user twists the clips 330, 332 after securing the guard 320 on the forceps 230, the user may grasp the forceps 230 and use it as a handle while twisting the barrier portion 322 relative to the first arm 228. Advantageously, the guard 320 is freely and quickly pivotable relative to the forceps 230 during any type of procedure, such as surgery or suturing, for example.

Digital pressure applied to the clips 330, 332 and to the barrier portion 322 creates a twisting force on the post 336. The twisting force causes the ratchet teeth 352 on the post 336 to bear against the ratchet teeth 350 on the jaws 338. The interengagement of the ratchet teeth 350, 352 causes the upper and lower jaws 342, 344 to flex away from one another. Above a threshold torsion force, the ratchet teeth 352 on the post 336 move clear of the ratchet teeth 350 on the jaws 338, enabling the post 336 to rotate. The elastic flexibility of the jaws 338 forces the ratchet teeth 350 on the jaws 338 back into engagement with the ratchet teeth 352 on the post 336. Thus, once the applied torsion force drops below the threshold necessary to cause rotation, the interengagement of the ratchet teeth 350, 352 once again maintains the angular position of the post 336 (and the clips 330, 332) relative to the barrier portion 322.

When using the guard 320 of FIGS. 30-35 to displace tissue, the tissue applies a reaction force to the barrier portion 322. If this reaction force is great enough, it could cause the barrier portion 322 to rotate about the first arm 228. Preferably, the guard 320 is designed to prevent such rotation under expected loads. To increase the torsion force necessary to rotate the barrier portion 322 about the first arm 228, for example, the ratchet teeth 350, 352 may be enlarged, or additional ratchet teeth 350, 352 may be added to other portions of the post 336. In the illustrated embodiment, only the rear portion of the post 336 adjacent the rear clip 332 includes ratchet teeth 352. Additional ratchet teeth 352 may be added to the portion of the post 336 that lies between the clips 330, 332, and/or to the portion of the post 336 that lies forward of the forward clip 330.

To help prevent unwanted rotation, the guard 320 of FIGS. 30-35 includes an anti-rotation clamp 354. With reference to FIGS. 30 and 32, the clamp 354 comprises a substantially L-shaped member residing atop a substantially flat wing 356 that extends from the left edge 334 of the barrier portion 322. The wing 356 extends along most of the left edge 334 of the barrier portion 322. However, those of skill in the art will appreciate that the wing 356 could be substantially shorter and still achieve the desired purpose. The clamp 354 is positioned adjacent the forward clip 330 and includes an overhanging lip 358 that faces the forward clip 330. However, those of skill in the art will appreciate that the clamp 354 could be positioned adjacent the rear clip 332.

The clamp 354 is positioned at a distance from the forward clip 330 such that when the clips 330, 332 are rotated into the position shown in FIG. 32, the clamp 354 captures the upper end of an outer wall of the forward clip 330. The lip 358 of the clamp 354 overlaps a portion of the upper end of the outer wall. Thus, when a force is applied to the barrier portion 322 that tends to rotate the barrier portion 322 toward the configuration shown in FIG. 30, the clamp 354 provides support tending to resist that rotation. To disengage the clamp 354 from the outer wall, the user may apply downward digital pressure on any part of the wing 356. A sufficient digital force flexes the wing 356 downward, moving the clamp 354 away from the forward clip 330. With the clamp 354 disengaged from the forward clip 330, the user may twist the clips 330, 332 relative to the barrier portion 322 as described above.

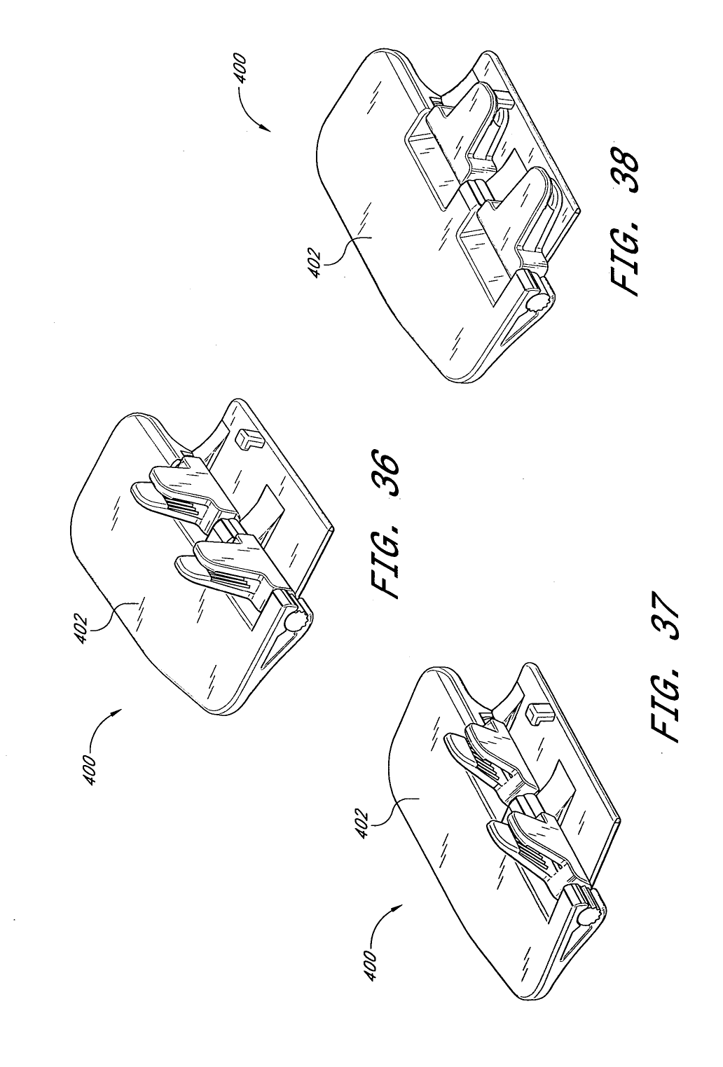

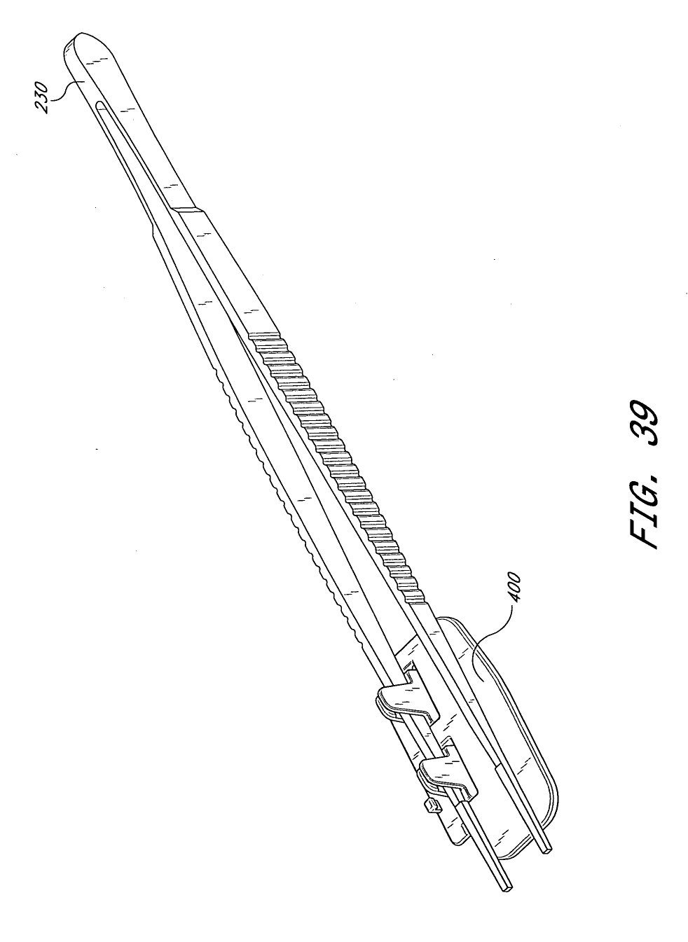

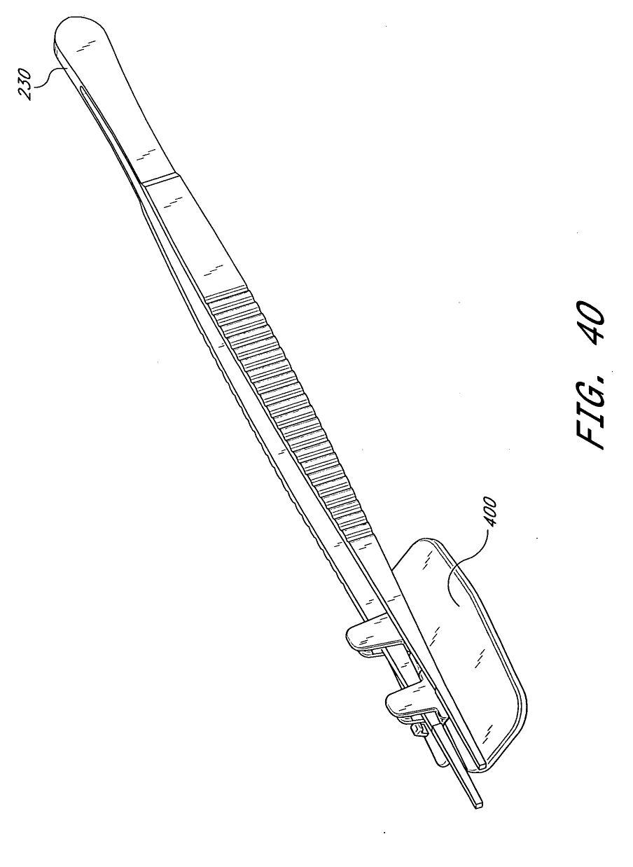

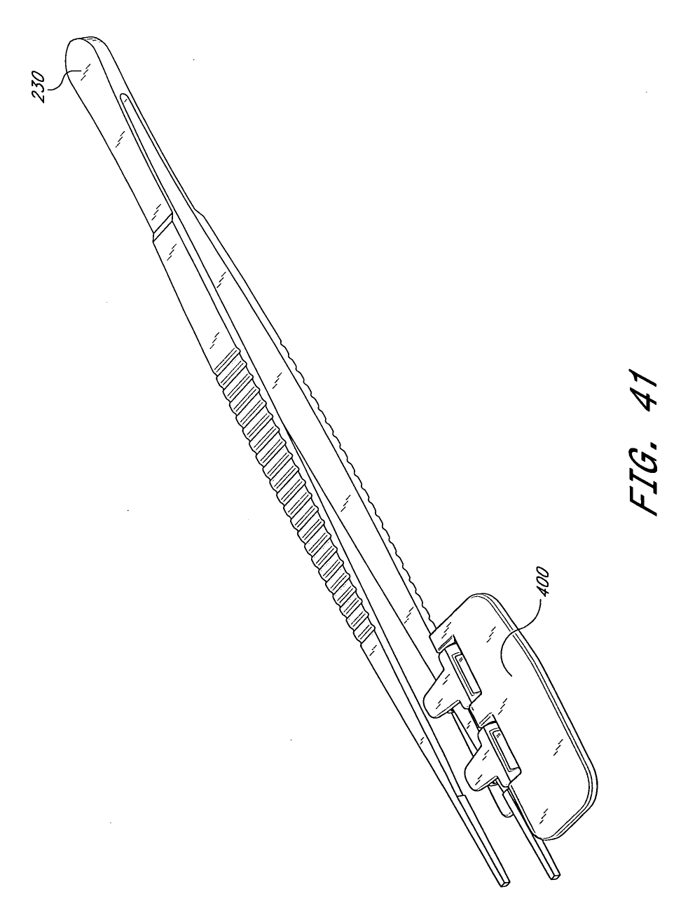

FIGS. 36-41 illustrate another preferred embodiment of the present surgical tissue guard. With reference to FIGS. 36-38, the guard 400 is substantially identical to the guard 320 illustrated in FIGS. 30-32. However, the guard 400 of FIGS. 36-38 includes a substantially flat barrier portion 402 with no curved portion. FIGS. 39-41 illustrate the guard 400 secured to the forceps 230, with the guard 400 pivoted in a variety of orientations relative to the forceps 230.

FIGS. 42-44 illustrate another preferred embodiment of the present surgical tissue guard. The guard 420 is substantially identical to the guard 320 illustrated in FIGS. 30-32. However, the guard 420 of FIGS. 42-44 does not include an anti-rotation clamp 354. FIGS. 42-44 illustrate various relative positions of the clips 422, 424 and the barrier portion 426.

Each of the guards described above is preferably constructed of a bio-compatible, rigid, puncture resistant material. However, the guard may be constructed of a semi-rigid material, or a combination of rigid and semi-rigid materials. Such semi-rigid materials are preferably able to flex a small amount. The material is preferably substantially transparent, so that a physician can see through the guard to the tissue that he or she is displacing or protecting with the guard. However, the guard may be constructed of an opaque material, or a combination of transparent and opaque materials. The material may be radio-transparent, or radio-opaque/radio-lucent, or a combination thereof. If the material is radio-opaque/radio-lucent, the guard will advantageously be visible from outside the patient's body. The guard can thus be easily recovered in case it is accidentally left inside the patient. The material should be non-toxic and safe for use internally. In order to facilitate easy attachment to and detachment from the forceps, the material is preferably flexible enough to allow the clips to flex as the forceps' first arm is inserted into the clips. Preferred materials include bio-compatible plastics, and polycarbonates, such as Lexan®, and metals such as titanium and stainless steel.

Those of skill in the art will appreciate that any embodiment of the present surgical tissue guard could be permanently attached to the forceps. For example, the tissue guard could be formed integrally with the forceps. Alternatively, the tissue guard could be formed separately from a typical forceps and then permanently attached to the forceps. Those of skill in the art will further appreciate that the configuration of any embodiment of the present surgical tissue guard may be constructed with a substantially reverse configuration from those shown. For example, the guard may be securable to the second arm of the forceps.

### Scope of the Invention

The above presents a description of the best mode contemplated for carrying out the present surgical tissue guard, and of the manner and process of making and using it, in such full, clear, concise, and exact terms as to enable any person skilled in the art to which it pertains to make and use this surgical tissue guard. This surgical tissue guard is, however, susceptible to modifications and alternate constructions from that discussed above that are fully equivalent. Consequently, this surgical tissue guard is not limited to the particular embodiments disclosed. On the contrary, this surgical tissue guard covers all modifications and alternate constructions coming within the spirit and scope of the surgical tissue guard as generally expressed by the following claims, which particularly point out and distinctly claim the subject matter of the surgical tissue guard.

1. A guard adapted to be attached to a forceps, comprising:
a barrier portion; and
at least one clip extending from the barrier portion, the clip including first and second spaced side walls.

2. The guard of claim 1, wherein upper ends of the first and second side walls are not connected to one another.

3. The guard of claim 1, wherein at least the first side wall includes a barb protruding from an inner surface thereof.

4. The guard of claim 1, wherein the barb is diagonally sloped.

5. The guard of claim 1, wherein the barrier portion includes rounded corners.

6. The guard of claim 1, wherein the barrier portion includes a main body portion, and at least a portion of the main body portion is shaped as a substantially flat plate.

7. The guard of claim 6, wherein a panhandle portion extends from an edge of the main body portion, the panhandle portion being substantially flat and of smaller surface area than the main body portion.

8. The guard of claim 6, wherein the main body portion includes a curved portion that extends forward from a forward portion of the flat plate portion and arches downward from the flat plate portion.

9. The guard of claim 1, further comprising a plurality of clips, the clips being aligned with one another such that an inner surface of each side wall lies in the same plane as at least one other side wall inner surface.

10. The guard of claim 1, wherein the at least one clip is fixed against rotation with respect to the barrier portion, and the at least one clip extends substantially perpendicularly to the barrier portion.

11. The guard of claim 1, wherein the at least one clip is fixed against rotation with respect to the barrier portion, and the at least one clip extends substantially parallel to the barrier portion.

12. The guard of claim 1, wherein the at least one clip is rotatable with respect to the barrier portion.

13. The guard of claim 12, wherein a first end of the at least one clip is secured to a post, and at least a portion of the post is captured between jaws of the barrier portion, and the post is rotatable within the jaws.

14. The guard of claim 13, wherein the jaws and the post include intermeshing ratchet teeth, the ratchet teeth being adapted to prevent relative rotation of the post and jaws except under application of a threshold torsion force.

15. The guard of claim 12, further comprising a clamp spaced from the at least one clip, the clamp being adapted to apply a force to the at least one clip to thereby resist rotation of the at least one clip.

16. The guard of claim 12, further comprising means to resist rotation of the at least one clip with respect to the barrier portion.

17. The guard of claim 1, wherein the clips are situated upon a raised portion of the barrier portion.

18. The guard of claim 1, wherein the guard is adapted to be attached to a first arm of a forceps such that the barrier portion extends toward a second arm of the forceps, and the second arm is freely movable across the barrier portion, the barrier portion being adapted to prevent tissue from getting between the arms from a side of the forceps to which the barrier is attached.

19. The guard of claim 1, wherein the guard is constructed of a substantially rigid material.

20. The guard of claim 1, wherein the guard is constructed of a substantially semi-rigid material.

21. The guard of claim 1, wherein the guard is constructed of a combination of substantially rigid and substantially semi-rigid materials.

22. The guard of claim 1, wherein the guard is constructed of a substantially transparent material.

23. The guard of claim 1, wherein the guard is constructed of a substantially opaque material.

24. The guard of claim 1, wherein the guard is constructed of a combination of substantially transparent and substantially opaque materials.

25. The guard of claim 1, wherein the guard is constructed of a substantially radio-transparent material.

26. The guard of claim 1, wherein the guard is constructed of a substantially radio-opaque or radio-lucent material.

27. The guard of claim 1, wherein the guard is constructed of a combination of substantially radio-transparent material and substantially radio-opaque or radio-lucent material.

28. The guard of claim 1, wherein the guard is constructed of a biocompatible material.

29. The guard of claim 1, wherein the guard is permanently secured to the forceps.

30. The guard of claim 29, wherein the guard is formed integrally with the forceps.

31. A method of displacing or protecting tissue during a surgical or suturing procedure, the method comprising the steps of:
attaching to a forceps a tissue guard, the guard including a barrier portion and at least one clip extending from the barrier portion; and
performing a surgical or suturing procedure.
